(19) United States
(12) Patent Application Publication
(10) Pub. No.: US 2023/0293490 A1
(43) Pub. Date: Sep. 21, 2023

(54) PROFILIN1:ACTIN INHIBITOR AS AN ANTI-ANGIOGENIC COMPOUND

(71) Applicants: University of Pittsburgh- Of the Commonwealth System of Higher Education, Pittsburgh, PA (US); Sorbonne Université, Paris (FR); Centre National de la Recherche Scientifque, Paris (FR); INSERM(Institut National de la Sante et de la Recherche Medicale), Paris (FR)

(72) Inventors: Partha Roy, Pittsburgh, PA (US); Lee McDermott, Pittsburgh, PA (US); Abigail E. Allen, Powell, OH (US); David Koes, Pittsburgh, PA (US); David M. Gau, Plum, PA (US); Paul G. Francoeur, Pittsburgh, PA (US); Xavier Guillonneau, Paris (FR)

(73) Assignees: University of Pittsburgh - Of the Commonwealth System of Higher Education, Pittsburgh, PA (US); Sorbonne Université, Paris (FR); Centre National de la Recherche Scientifique, Paris (FR); INSERM (Institut de la Sante er de la Recherche Medicale), Paris (FR)

(21) Appl. No.: 18/019,219

(22) PCT Filed: Aug. 12, 2021
(86) PCT No.: PCT/US2021/045706

§ 371 Date: Feb. 1, 2023

Related U.S. Application Data
(60) Provisional application No. 63/066,709, filed on Aug. 17, 2020.

(51) Int. Cl. A61K 31/4155; A61P 35/00

(52) U.S. Cl. CPC A61K 31/4155; A61P 35/00

(57) ABSTRACT

Disclosed are anti-angiogenic agents that target Pfn1:actin interaction. Also disclosed are compounds for treating ocular diseases or conditions. Further disclosed are compounds for treating cancer.

### CROSS REFERENCE TO RELATED APPLICATIONS

This claims the benefit of U.S. Provisional Application No. 63/066,709, filed Aug. 17, 2020, which is incorporated by reference herein.

### ACKNOWLEDGMENT OF GOVERNMENT SUPPORT

This invention was made with government support under grant numbers GM108340, CA108607, and TR001857 awarded by the National Institutes of Health, and under grant number W81XWH-19-1-0768 awarded by the Department of Defense. The government has certain rights in the invention.

### BACKGROUND

Profilin(Pfn1)-actin interaction is critical for actin-driven biological processes including angiogenesis. This interaction is of particular interest in treatment of aberrant angiogenesis such as in the retina in pathologies like proliferative diabetic retinopathy (PDR), wet age-related macular degeneration (AMD), retinopathy of prematurity (ROP) and certain types of cancer (in particular, clear cell renal cell carcinoma). While anti-angiogenic therapies targeting VEGF signaling are generally effective in some of these disease contexts, spontaneous or acquired resistance to anti-VEGF therapies continues to be significant problem in many of these diseases.

The estimated incidence of and number of deaths from renal cell carcinoma (RCC) in the United States in 2019 are 73,280 and 14,770, respectively. The most common subtype, clear cell RCC (ccRCC), occurs in >75% of RCC patients. Approximately 20-30% of those patients present with metastasis at the time of diagnosis. Another one-third of patients, following initial treatment, develop either local recurrence and/or distant metastases. Disquietingly, the five-year survival of patients with advanced stage ccRCC remains only 10%. A distinguishing hallmark of ccRCC is its highly vascularized tumor microenvironment (TME) arising from the genetic loss-of-function (LOF) of the tumor suppressor protein Von Hippel Lindau (VHL—inactivated in >90% of cases of sporadic ccRCC) leading to hypoxia-inducible factors-1 and 2 (HIF1/2) stabilization and upregulation of the pro-angiogenic factor, VEGF. While many ccRCC patients initially respond to therapies targeting VEGF or other proangiogenic signaling pathways, very few patients exhibit durable treatment-associated benefits and virtually all develop progressive, drug-refractory disease.

Aberrant angiogenesis also lies at the heart of a wide range of ocular pathologies such as proliferative diabetic retinopathy (PDR), wet age-related macular degeneration (AMD) and retinopathy of prematurity. In particular, PDR is one of the leading causes of blindness worldwide. While intravitreal anti-angiogenic therapies targeting vascular endothelial growth factor (VEGF) signaling are generally effective in some of these disease contexts, spontaneous or acquired resistance to anti-VEGF therapies continues to be significant problem in a substantial percentage of the patient population (˜30% in PDR and ˜10% in wet AMD) because of involvement of other pro-angiogenic mediators in disease progression. Hence, identification of alternative mediators of retinal angiogenesis could lead to novel therapeutics for ocular diseases.

### SUMMARY

Disclosed herein in one embodiment are compounds, or pharmaceutically acceptable salts thereof, of formula I:

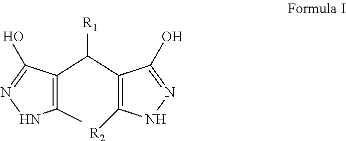

wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and R₂ is hydrogen, alkyl, substituted alkyl, cycloalkyl, or substituted cycloalkyl,

provided that the compound is not

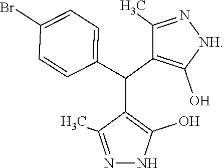 [SMILES: Cc1n[nH]c(O)c1C(c1ccc(Br)cc1)c1c(C)n[nH]c1O]

Also disclosed herein in another embodiment are compounds, or pharmaceutically acceptable salts thereof, of formula II:

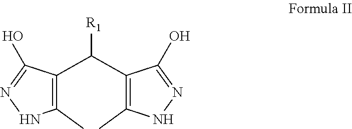

wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl, provided that the compound is not

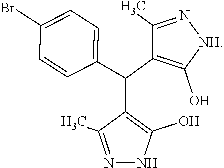 [SMILES: Cc1n[nH]c(O)c1C(c1ccc(Br)cc1)c1c(C)n[nH]c1O]

Further disclosed herein in another embodiment are compounds, or pharmaceutically acceptable salts thereof, of formula III:

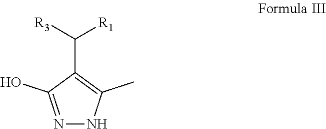

wherein R₁ is aryl, substituted aryl, heteroaryl, substituted heteroaryl, alkyl, substituted alkyl, 10 heterocycloalkyl, substituted heterocycloalkyl, cycloalkyl, or substituted cycloalkyl; and R₃ is N-heterocyclic group or substituted N-heterocyclic group, provided that the compound is not

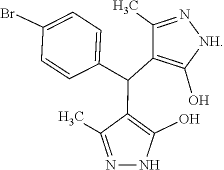 [SMILES: Cc1n[nH]c(O)c1C(c1ccc(Br)cc1)c1c(C)n[nH]c1O]

Disclosed herein in another embodiment is a method for treating an angiogenesis-dependent disease or condition in a subject, comprising administering to the subject in need thereof a therapeutically effective amount of a compound.

Disclosed herein in another embodiment is a method for treating an ocular disease or condition in a subject, comprising administering to the subject in need thereof a therapeutically effective amount of a compound.

Disclosed herein in another embodiment is a method for treating cancer in a subject, comprising administering to the subject in need thereof a therapeutically effective amount of a compound.

Disclosed herein in another embodiment is a method for inhibiting migration and/or proliferation of renal cell carcinoma cells, comprising contacting renal cell carcinoma cells with a compound.

Disclosed herein in another embodiment is a method for inhibiting ocular neovascularization in a subject, comprising administering to the subject in need thereof a therapeutically effective amount of a compound.

Disclosed herein in another embodiment is a method for inhibiting tumor neovascularization in a subject, comprising administering to the subject in need thereof a therapeutically effective amount of a compound.

Disclosed herein in another embodiment is a method for inhibiting tumor growth in a subject, comprising administering to the subject in need thereof a therapeutically effective amount of a compound.

The foregoing and other objects, features, and advantages will become more apparent from the following detailed description, which proceeds with reference to the accompanying figures.

### BRIEF DESCRIPTION OF THE DRAWINGS

FIGS. 1A-1B. Adverse association of Pfn1 with patient outcome in human ccRCC. A-B) Association of transcript expressions of Pfn1 with disease features (grade, stage, metastasis) (panel A) and survival (OS and PFS) (panel B) of ccRCC patients based on the analyses of TCGA data representing 537 clinical cases. For survival analyses, transcript expression was

dichotomized at the median value. Red and blue lines in Kaplan-Meir survival plots indicate higher and lower than median expression, respectively.

FIGS. 2A-2B: Expression of Pfn1 in cells within the TME of human ccRCC. A) Pfn1 IHC of ccRCC TMAs showing % frequency of tumors with different expression patterns of Pfn1 (N=417 tumors); (blue—nuclei) [courtesy: Stefan Duensing lab, Heidelberg School of Medicine]. B) Transcript expression profiling of Pfn1 and a few other actin-binding proteins (Mena and CapZ isoforms) in CD34+CD45negCD146+ VEC flow-sorted from the digests of fresh renal tumors and patient-matched normal adjacent tissues (N=8 patients) [courtesy: Walter Storkus lab, University of Pittsburgh School of Medicine].

FIGS. 3A-3G: Pfn1 promotes migration and proliferation of RCC cells. Effect of Pfn1 KD on migration and proliferation of VHL-negative murine RCC (RVN) (panels A-D) and human RCC (786-0) (panels E-G) cells. Pfn1 immunoblots in panels A and E demonstrate Pfn1 KD (tubulin blot serves as the loading control). Data is representative of that obtained from three independent experiments performed (*:p<0.05, **p<0.01).

FIGS. 4A-4F: Effect of modulating Pfn1 expression in VEC on RCC cell migration. A) Pfn1 and GAPDH (loading control) immunoblots showing the effect of adenoviral-mediated Pfn1 overexpression in VEC on extracellular release of Pfn1 as measured in the conditioned media (Ad-GFP: control). B) Effect of purified rPfn1 protein in culture media on the average speed of VEC and RCC in random motility assay (data normalized to control with buffer only; N=40 cells per group from 2 independent experiments). C-F) Effects of conditioned media of Pfn1 overexpressing (panel C-D) and KD (panels E-F) cultures of VEC on chemotactic migration of RVN cells (migration data normalized to control adenovirus transduction or siRNA transfection condition). Pfn1 and GAPDH (loading control) immunoblots in panels C and E confirm Ad-Pfn1- and Pfn1-siRNA-mediated overexpression and KD of Pfn1, respectively (data summarized from 3 experiments). In the bar graphs, * and ** denote p<0.05 and p<0.01, respectively.

FIGS. 5A-5D. Demonstration of C74's ability to reduce RCC cell proliferation in vitro and tumor growth in vivo. A-B) Effect of indicated doses of C74 on proliferation and serum-induced chemotactic migration of RVN cells (**: p<0.01; ***: p<0.001). C-D) Effect of in vivo administration of C74 vs DMSO on tumor growth (panel C—representative tumors, panel D—quantification) from subcutaneously implanted RENCA cells in Balb/c mice (data summarized from 9 and 10 DMSO- and C74-treated mice pooled from two independent experiments).

FIG. 6. General synthesis schemes and methods for compounds disclosed herein. Synthesis of compounds of the general structure 4-Symmetric may be effected via the reaction of 2 eq of a pyrazolone with 1 eq of a desired aldehydes in a Knoevenagel, Michael tandem reaction sequence under conditions known in the literature and to the persons skilled in the art (Wang W, et al. Synthetic Communications. 2005, 35(9), 1263; Vafaee A, et al. Research on Chemical Intermediates. 2015, 41(11), 8343). Compounds of the general structure 4-Symmetric can also be synthesized in a multicomponent reaction format in which an aldehyde, 2 eq. of hydrazine or hydrazine hydrochloride and 2 eq. of ethyl acetoacetate react in one pot under conditions and methods known to the persons skilled in art (Soleimani E, et al. Comptes Rendus Chimie 2012, 15(11-12), 955; Jia, X.-D. et al. Chemical Research in Chinese Universities 2012, 6, 999). Compounds of the general structure 4-Asymmetric may be produced via the Knoevenagel condensation products 3 that in turn may be prepared by reaction of a desired aldehyde and 1 eq. of methylpyrazolone (Bule, S. et al. American Journal of Pharmacy and Health Research 2013, 1 (6), 47) followed by a Michael reaction with a second differently substituted pyrazolone. Analogs of the general structure 7 in which one of the hydroxypyrazoles has been replaced by another moiety may be prepared through a combination of known literature protocols. Ketones of the general structure 5, may react in a Knoevenagel fashion with pyrazolones to afford Knoevenagel condensation products of general structure 6 that in turn may be reduced with NaBH₄ or Hantzsch ester hydride (HEH) or other suitable reducing agent to the desired compounds (Liu, Z. et al Synlett. 2005, 10, 1579; Reddy, G. et al. Indian Journal of Chemistry 2019, 58B, 1042). Alternatively, such derivatives may be prepared by the reaction of ethylacetoacetate with appropriate substrates of the general structure 8 to provide ketoesters 9 which in turn may be converted to desired derivatives by treatment with hydrazine.

FIGS. 7A-7B. Pfn1-dependency for retinal angiogenesis: A) Representative images of whole mount FITC-lectin stained P6 retinae from Pfn1^{WT} (wildtype) and Pfn1^{KO} mice along with quantification of retinal VEC (vascular EC) coverage area. B) Representative close-up images of the growing vascular front in Pfn1^{WT} vs Pfn1^{KO} retinae along with quantifications of sprouts (per 20× field) and vascular density in the two groups showing severe hypovascularization in Pfn1^{KD} retinae (n=12 retinae per group).

FIGS. 8A-8D. A-B) Images (panel A) of choroidal explant angiogenesis culture on D6 under vehicle (DMSO) and C74-treated conditions (inset shows explants on D3; red outline shows the edge of the vascular outgrowth); Panel B shows quantification. Laser-induced choroidal NV (CNV—an in vivo model that induces sub-retinal NV in response to laser injury in mice and is widely used to study NV aspects of wet AMD) in the settings of IVT injection of either DMSO (VEH) or C74 (50 μM) (panel C) and associated quantification (panel D).

FIGS. 9A-B. A) Pyrene-actin polymerization assays (fluorescence measures actin polymerization) show that C74 can reverse Pfn1's inhibition of actin polymerization (A—actin, P-Pfn1, the numbers in the parentheses indicate relative stoichiometry). Note that contrasting its effect in cells, in biochemical assay Pfn1 inhibits actin polymerization by actin sequestering, and neither compound affects actin polymerization kinetics without the presence of Pfn1 (an important screening criterion). B) Demonstration of anti-angiogenic action of compound C74 on human dermal VEC in a dose-dependent manner in matrigel cord assay in vitro with C74 being able to inhibit angiogenesis at ˜5-fold lower concentration (10 μM) than C2 (50 μM).

FIG. 10 shows that daily intratumoral administration of C74 reduces tumor angiogenesis (marked by lower CD31-positive immune-reactive region of tumor histosections) in subcutaneous tumors established by RENCA cells in Balb/c mice, relative to DMSO (vehicle) control.

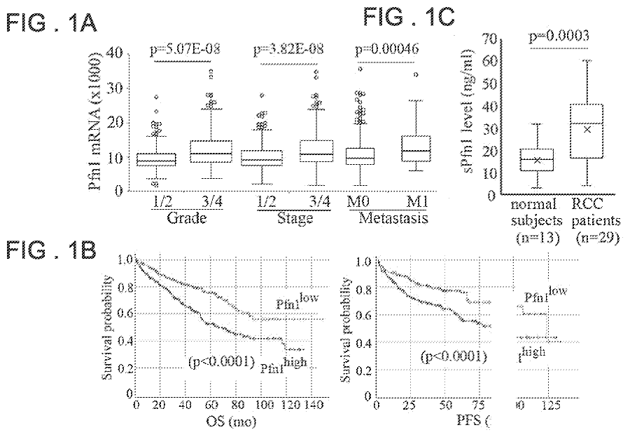

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Administration” as used herein is inclusive of administration by another person to the subject or self-administration by the subject.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1- hexenyl, 2-hexenyl, 3-hexenyl, 4-hexenyl, or 5-5 hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

An “analog” is a molecule that differs in chemical structure from a parent compound, for example a homolog (differing by an increment in the chemical structure or mass, such as a difference in the length of an alkyl chain or the inclusion of one of more isotopes), a molecular fragment, a structure that differs by one or more functional groups, or a change in ionization. An analog is not necessarily synthesized from the parent compound. A derivative is a molecule derived from the base structure.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals. Illustrative non-human mammals include animal models (such as mice), non-human primates, companion animals (such as dogs and cats), livestock (such as pigs, sheep, cows), as well as non-domesticated animals, such as the big cats. The term subject applies regardless of the stage in the organism's lifecycle. Thus, the term subject applies to an organism in utero or in ovo, depending on the organism (that is, whether the organism is a mammal or a bird, such as a domesticated or wild fowl).

“Aryl” refers to a monovalent unsaturated aromatic carbocyclic group having a single ring (e.g., phenyl) or multiple condensed rings (e.g., naphthyl or anthryl), which can optionally be unsubstituted or substituted. A “heteroaryl group,” is defined as an aromatic group that has at least one heteroatom incorporated within the ring of the aromatic group. Examples of heteroatoms include, but are not limited to, nitrogen, oxygen, sulfur, and phosphorous. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The aryl or heteroaryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl or heteroaryl group can be unsubstituted.

The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, and the like. The term “heterocycloalkyl group” is a cycloalkyl group as defined above where at least one of the carbon atoms of the ring is substituted with a heteroatom such as, but not limited to, nitrogen, oxygen, sulfur, or phosphorous.

The terms “halogenated alkyl” or “haloalkyl group” refer to an alkyl group with one or more hydrogen atoms present on these groups substituted with a halogen (F, Cl, Br, I).

“Inhibiting” refers to inhibiting the full development of a disease or condition. “Inhibiting” also refers to any quantitative or qualitative reduction in biological activity or binding, relative to a control.

“N-heterocyclic” refers to mono or bicyclic rings or ring systems that include at least one nitrogen heteroatom. The rings or ring systems generally include 1 to 9 carbon atoms in addition to the heteroatom(s) and may be saturated, unsaturated or aromatic (including pseudoaromatic). The term “pseudoaromatic” refers to a ring system which is not strictly aromatic, but which is stabilized by means of delocalization of electrons and behaves in a similar manner to aromatic rings. Aromatic includes pseudoaromatic ring systems, such as pyrrolyl rings.

Examples of 5-membered monocyclic N-heterocycles include pyrrolyl, H-pyrrolyl, pyrrolinyl, pyrrolidinyl, oxazolyl, oxadiazolyl, (including 1,2,3 and 1,2,4 oxadiazolyls) isoxazolyl, furazanyl, thiazolyl, isothiazolyl, pyrazolyl, pyrazolinyl, pyrazolidinyl, imidazolyl, imidazolinyl, triazolyl (including 1,2,3 and 1,3,4 triazolyls), tetrazolyl, thiadiazolyl (including 1,2,3 and 1,3,4 thiadiazolyls), and dithiazolyl. Examples of 6-membered monocyclic N-heterocycles include pyridyl, pyrimidinyl, pyridazinyl, pyrazinyl, piperidinyl, morpholinyl, thiomorpholinyl, piperazinyl, and triazinyl. The heterocycles may be optionally substituted with a broad range of substituents, and preferably with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino. The N-heterocyclic group may be fused to a carbocyclic ring such as phenyl, naphthyl, indenyl, azulenyl, fluorenyl, and anthracenyl.

Examples of 8, 9 and 10-membered bicyclic heterocycles include 1H thieno[2,3-c]pyrazolyl, indolyl, isoindolyl, indolinyl, azaindolyl, benzoxazolyl, benzothiazolyl, benzisoxazolyl, benzisothiazolyl, benzimidazolyl, indazolyl, isoquinolinyl, quinolinyl, quinoxalinyl, purinyl, cinnolinyl, phthalazinyl, quinazolinyl, quinoxalinyl, benzotriazinyl, and the like. These heterocycles may be optionally substituted, for example with C₁₋₆ alkyl, C₁₋₆ alkoxy, C₂₋₆ alkenyl, C₂₋₆ alkynyl, halo, hydroxy, mercapto, trifluoromethyl, amino, cyano, oxo or mono or di(C₁₋₆alkyl)amino. Unless otherwise defined optionally substituted N-heterocyclics includes pyridinium salts and the N-oxide form of suitable ring nitrogens.

Small organic molecule: An organic molecule with a molecular weight of about 1000 daltons or less (for example about 900 daltons or less, about 800 daltons or less, about 700 daltons or less, about 600 daltons or less, about 500 daltons or less, about 400 daltons or less, about 300 daltons or less, about 200 daltons or less, or about 100 daltons or less). In some examples, a small organic molecule has a molecular weight of about 100-1000 daltons, about 200-900 daltons, about 300-700 daltons, about 200-500 daltons, or about 400-700 daltons.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's lifecycle. Thus, the term subject applies to an organism in utero or in ovo, depending on the organism (that is, whether the organism is a mammal or a bird, such as a domesticated or wild fowl).

“Substituted” or “substitution” refers to replacement of a hydrogen atom of a molecule or an R-group with one or more additional R-groups. Unless otherwise defined, the term “optionally-substituted” or “optional substituent” as used herein refers to a group which may or may not be further substituted with 1, 2, 3, 4 or more groups, preferably 1, 2 or 3, more preferably 1 or 2 groups. The substituents may be selected, for example, from C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃ and CHF₂), C₁₋₆alkoxyhalo (such as OCF₃ and OCHF₂), carboxyl, esters, cyano, nitro, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case N-heterocycles may also include but are not limited to C₁₋₆alkyl i.e. N—C₁₋₃alkyl, more preferably methyl particularly N-methyl.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. F or a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs may include compounds having a phosphonate, hydroxy, thio and/or amino group functionalized with any group that is cleaved in vivo to yield the corresponding amino, hydroxy, thio and/or phosphonate group, respectively. Examples of prodrugs can include, without limitation, compounds having an acylated amino group and/or a phosphonate ester or phosphonate amide group.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Particular examples of the presently disclosed compounds may include one or more asymmetric centers; thus the compounds described can exist in different stereoisomeric forms. Accordingly, compounds and compositions may be provided as individual pure enantiomers or as stereoisomeric mixtures, including racemic mixtures. In certain embodiments the compounds disclosed herein may be synthesized in or may be purified to be in substantially enantiopure form, such as in a 90% enantiomeric excess, a 95% enantiomeric excess, a 97% enantiomeric excess or even in greater than a 99% enantiomeric excess, such as in enantiopure form.

The presently disclosed compounds can have at least one asymmetric center or geometric center, cis-trans center(C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also include all isotopes of atoms present in the compounds, which can include, but are not limited to, deuterium, tritium, ¹⁸F, etc.

### Overview

Dynamic control of the actin cytoskeleton is a key feature of all actin-dependent biological processes that include cell migration and proliferation in both physiological and pathological contexts. Actin cytoskeletal regulation in cells involves the concerted actions of several major classes of actin-binding proteins (ABPs). Among them, the profilin (Pfn) family of ABPs play a key role in promoting actin polymerization in cells through their nucleotide-exchange activity on G-actin (facilitates ADP-to-ATP exchange on G-actin) and ability to act as a carrier of ATP-G-actin to a number of other actin assembly factors bearing poly-proline (PLP) motifs during F-actin elongation. Pfn's importance in the regulation of actin dynamics, and actin-based cellular processes, such as cell migration and proliferation, is well-established in the literature. Differential proteomic analyses of supernatants from the cultures of RCC cells vs normal kidney epithelial cells, supported by RT-PCR and qualitative immunohistochemistry (IHC) analyses of a small number of RCC (14 cases) vs normal kidney tissues provided the first evidence of overexpression of Pfn1 (the major isoform of Pfn family of ABPs) in human ccRCC. Proteomic studies further identified Pfn1 to be one of the candidate markers of late stage ccRCC. In concordance with these findings, semi-quantitative IHC studies performed with large cohort of patient samples (384 cases) subsequently established higher Pfn1 expression correlated with shorter overall survival (OS) and progression-free survival (PFS) of ccRCC patients. Collectively, these studies suggest Pfn1's positive association with advanced disease features and adverse clinical outcomes in the setting of ccRCC, findings further corroborated by us by TCGA transcriptome data analyses of ccRCC patient cohorts. We further demonstrate dysregulated expression of Pfn1 and a subset of other ABPs queried herein in tumor-associated VEC (TA-VEC). Additional experimental evidence support the role of Pfn1 in proliferation and migration of RCC cells, and VEC-tumor cell crosstalk. Disclosed herein is a Pfn1:actin interaction inhibitor that reduces RCC cell aggressiveness in vitro and tumor growth in vivo.

Disclosed herein are several novel findings. Specifically, we for the first time provide evidence for: i.) dramatic Pfn1 upregulation in TA-VEC in human ccRCC, ii.) the ability of Pfn1 to modulate RCC tumor cell migration in both intrinsic and extrinsic (i.e. paracrine) formats, and iii) diminished aggressiveness of RCC cells in response to a small molecule inhibitor of the Pfn1:actin interaction. Collectively, these novel findings suggest that Pfn1 may represent not only a cogent tissue biomarker of clinical prognosis in ccRCC patients, but a potential interventional target to limit tumor progression.

Disclosed herein are small molecule inhibitors of the Pfn1:actin interaction and their ability to substantially reduce migration and proliferation of RCC cells in cell culture settings. Our studies demonstrate the ability of a representative inhibitor to reduce tumor growth in vivo.

Consistent with the important role of Pfn1 in regulating actin polymerization and various fundamental actin-based cellular activities (migration and proliferation), treatment with a representative of the disclosed compounds reduced the overall level of cellular filamentous (F) actin, slowed EC migration and proliferation, and inhibited the angiogenic ability of ECs both in vitro and in vivo.

Vascular endothelial cell (VEC) migration is a fundamental aspect of angiogenesis and is driven by dynamic remodeling of the actin cytoskeleton in response to pro-angiogenic stimuli. Along this line, we have shown that actin-binding protein (ABP) profilinl (Pfn1), a major regulator of actin dynamics and an important mediator of VEC migration and proliferation, plays an indispensable role in sprouting angiogenesis in vitro, ex vivo and in vivo in a mouse model of retinal angiogenesis. Our bioinformatics analyses provided further evidence for dramatically elevated Pfn1 expression in retinal VEC in individuals with PDR. Disclosed herein are small molecule antagonists of the Pfn1-actin interaction that substantially inhibit VEC migration and proliferation (key aspects of angiogenesis) without cytotoxicity and, accordingly, diminish angiogenesis in vitro, ex vivo and in vivo settings.

### Compounds

Disclosed herein are compounds, or pharmaceutically acceptable salts thereof, of formula I:

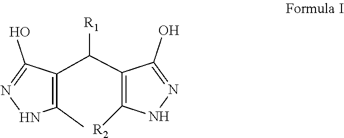

wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and R₂ is hydrogen, alkyl, substituted alkyl, cycloalkyl, or substituted cycloalkyl.

Also disclosed herein are compounds, or pharmaceutically acceptable salts thereof, of formula II:

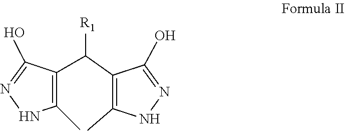

wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl.

Further disclosed herein are compounds, or pharmaceutically acceptable salts thereof, of formula III:

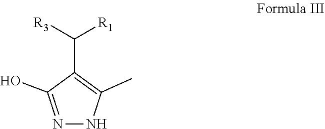

wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and

R₃ is N-heterocyclic group or substituted N-heterocyclic group.

Illustrative groups for R₁ include but are not limited to halogenated alkyl (e.g., —CF₃,), cyclopropyl, optionally substituted alkyl, phenyl, aryl (e. g. alkoxy phenyl, bromophenyl), optionally substituted heteroaryl (e.g. furanyl, thiophenyl), and optionally substituted heterocycloalkyl (e.g. tetrahydrofuranyl, tetrahydropyranyl, piperidinyl).

Illustrative groups for R₃ include but are not limited to indolyl, azaindolyl, pyridinyl, and hydroxypyrazolyl.

One compound is (4-[(4-bromophenyl)(5-hydroxy-3-methyl-1H-pyrazol-4-yl)methyl]-3-methyl-1H-pyrazol-5-ol) (referred to herein as C74) is shown below:

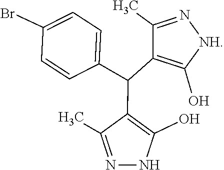 [SMILES: Cc1n[nH]c(O)c1C(c1ccc(Br)cc1)c1c(C)n[nH]c1O]

### Methods of Use and Pharmaceutical Compositions

In certain embodiments, the compounds are anti-angiogenic agents.

In certain embodiments, the compounds target Pfn1:actin interaction to inhibit a fundamental process for vessel formation.

In certain embodiments, the subject is in need of, or has been recognized as being in need of, treatment with an anti-angiogenic agent. The subject may be selected as being amenable to treatment with an anti-angiogenic agent. For example, the subject may be in need of an agent that inhibits angiogenesis caused by pro-angiogenic growth factor upregulation and tissue injury.

In certain embodiments, the compounds disclosed may be used for treating or preventing angiogenesis-dependent diseases or conditions. Illustrative diseases or conditions include those characterized by aberrant angiogenesis in the retina.

In certain embodiments, the compounds disclosed herein may be used to treat ocular diseases or conditions. Illustrative ocular diseases or conditions include proliferative diabetic retinopathy (PDR), wet age-related macular degeneration (AMD), retinopathy of prematurity (ROP), or angiogenesis-associated with eye injuries

In certain embodiments, the compounds may be used for treating cancer. Illustrative cancers include renal cell carcinoma (e.g., clear cell renal cell carcinoma), brain cancer and breast cancer.

In certain embodiments, the compounds inhibit ocular neovascularization.

In certain embodiments, the compounds inhibit tumor neovascularization.

In certain embodiments, the compounds inhibit tumor growth.

C74 demonstrated an ability to reverse Pfn1's effect on actin polymerization in an in vitro polymerization assay. C74 also inhibited angiogenic capability of human retinal endothelial cells in vitro and chorodial neovascularization ex vivo and in vivo (in laser induced choroidal neovascularization model—an in vivo experimental model for wet AMD). Furthermore, C74 reduced mouse and human renal cancer cell line proliferation and migration in a dose dependent manner. In a murine tumor model, preliminary studies suggest that daily injection of C74 resulted in decreased tumor weight over the course of the experiment.

In some embodiments, the methods disclosed herein involve administering to a subject in need of treatment a pharmaceutical composition, for example a composition that includes a pharmaceutically acceptable carrier and a therapeutically effective amount of one or more of the compounds disclosed herein. The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

In some embodiments, one or more of the disclosed compounds (including compounds linked to a detectable label or cargo moiety) are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, PA, 21^{st} Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The disclosed compounds and/or compositions can be enclosed in multiple or single dose containers. The compounds and/or compositions can also be provided in kits, for example, including component parts that can be assembled for use. For example, one or more of the disclosed compounds may be provided in a lyophilized form and a suitable diluent may be provided as separated components for combination prior to use. In some examples, a kit may include a disclosed compound and a second therapeutic agent (such as an anti-retroviral agent) for co-administration. The compound and second therapeutic agent may be provided as separate component parts. A kit may include a plurality of containers, each container holding one or more unit dose of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

In some examples, about 0.1 mg to 1000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 1000 mg (for example, about 2 mg to about 500 mg, about 5 mg to 50 mg, about 10 mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants. If oral administration is desired, the compound is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the compounds can be administered orally to humans in a dosage range of 1 to 1000 mg/kg body weight in single or divided doses. One illustrative dosage range is 0.1 to 200 mg/kg body weight orally (such as 0.5 to 100 mg/kg body weight orally) in single or divided doses. For oral administration, the compositions may be provided in the form of tablets containing about 1 to 1000 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

It should be apparent to one skilled in the art that the exact dosage and frequency of administration will depend on the particular compounds administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### EXAMPLES

### Pfn1 is a Marker of Poor Prognosis in ccRCC

We first queried a TCGA dataset representing 537 clinical cases of ccRCC to demonstrate that higher (dichotomized at median) mRNA expression of Pfn1 (but not the minor isoform Pfn2) correlated with disease features of advanced-stage tumors (higher tumor grade and stage, metastatic propensity) and lower overall survival (OS) as well as progression-free survival (PFS) of ccRCC patients, but not in patients with other histological subtypes of RCC (papillary and chromophobe).

### Pfn1 is Preferentially Overexpressed in Tumor-Associated Vascular Endothelial Cells (TA-VEC) in ccRCC

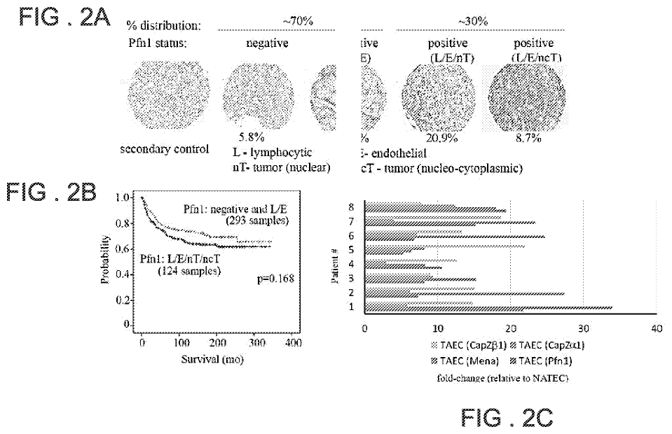

To determine which cells in the ccRCC tumor microenvironment (TME) preferentially overexpressed Pfn1, our collaborating lab (Dr. Stefan Duensing, Heidelberg School of Medicine) next performed a qualitative IHC assessment of tissue microarrays (TMAs) containing a large cohort (N=417) of ccRCC tumors (FIG. 2A). Interestingly, we observed 3 distinct patterns of Pfn1 expression: i.) negligible Pfn1 expression throughout the tumor (5.8%), ii.) strong Pfn1 expression in stromal cells (VEC, lymphocytes) but negligible expression in tumor cells (64.5%), and iii.) strong Pfn1 expression in both stromal and tumor cells (29.7%). In the latter sample cohort, 71% of specimens exhibited a nuclear distribution of Pfn1 expression in tumor cells, with the remaining 29% of specimens characterized by nucleocytoplasmic staining of tumor cells. These findings suggest that the vast majority of ccRCC tumors display a pattern of strong Pfn1 expression selectively in stromal cells within the TME.

Given the prevalence of a stromal cell-associated signature for Pfn1 overexpression in ccRCC, our collaborating lab (Dr. Stefan Duensing, Heidelberg School of Medicine) next performed gene expression profiling of flow-sorted CD34+CD45negCD146+ VEC isolated from freshly isolated enzymatically-digested, ccRCC tumors, which revealed a robust (5.3 to 21.7-fold) upregulation in Pfn1 expression in tumor-associated TA-VEC relative to VEC sorted from patient-matched normal adjacent tissue (NAT-VEC; FIG. 2C). Among the other poor prognostic cytoskeletal markers, only Mena, CapZα1 and CapZβ (also pre-determined from TCGA data analyses—data not shown) were also determined to be strongly upregulated in TAVEC vs. NAT-VEC (FIG. 2C). In contrast, a parallel analysis of flow-sorted CD34negCD45negCD146+ vascular pericytes isolated from tumor vs. NAT samples showed no significant differences in the expression of these proteins. Since Pfn1 interacts with Mena/VASP proteins and is a major promoter of Mena/VASP-mediated actin assembly and actin-driven cellular processes, transcriptional dysregulation of the Pfn1:Mena/VASP cytoskeletal pathway in TA-VEC could represent a bio-signature of poor prognosis in ccRCC.

### Pfn1 is an Important Regulator of RCC Tumor Cell Migration and Proliferation

Pfn1's interactions with actin and other actin assembly factors (such as Mena/VASP proteins) play important roles in actin polymerization at the leading edge and membrane protrusion (a key step of cell migration). In most physiological contexts, LOF of Pfn1 leads to defects in membrane protrusion and cell migration. However, in certain cancer cells (breast,

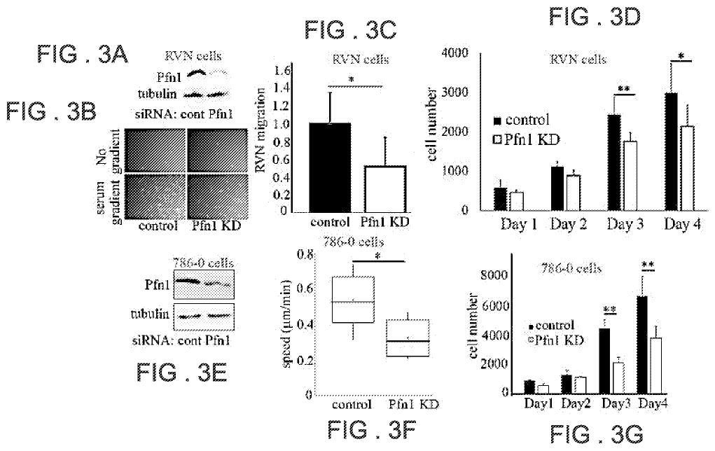

hepatic), Pfn1 depletion induces a hyper-migratory phenotype, suggesting that Pfn1's role in cell migration is highly context-specific. To determine whether Pfn1 has a pro- or anti-migratory effect in RCC cells, we first studied the effect of silencing Pfn1 expression on chemotactic and random migration of RVN (a variant of the murine RENCA RCC cell line engineered for VHL deletion by CRISPR/Cas9) and 786-0 (a VHL negative human cell line originally derived from ccRCC clinical specimen) cell lines, respectively. Note that although the parent RENCA (VHLWT) cell line has been considered poorly reflective of human ccRCC, RVN (VHL-/-) cells overexpress many of the genes associated with aggressive ccRCC in humans and appears to represent an appropriate surrogate of human disease. When implanted into syngeneic immunocompetent Balb/c mice, develop progressively growing tumors that metastasize throughout the body mimicking advanced stage disease in humans. We found that Pfn1 depletion leads to reduced migration of both RVN and 786-0 RCC cell lines (FIGS. 3A-C, E-F). Likewise, proliferation of both VHL -/- cell lines was reduced when Pfn1 expression was suppressed (FIGS. 3D, G). Collectively, these findings support a Pfn1-dependency for RCC migration and proliferation. Bi-directional communication between VEC and cancer cells through various cell surface receptors and soluble factors impacts the migratory behavior of cancer cells. Although Pfn1 is mainly an intracellular protein, the presence of Pfn1 in the conditioned media of various types of cultured cells (VEC, glomerular mesangial cells, RCC and breast cancer cells) has been documented previously by us and other groups. We have also shown that extracellular release of Pfn1 is sensitive to perturbations of actin-sensitive signaling pathways, suggesting that Pfn1 release from cells is an actively regulated process.

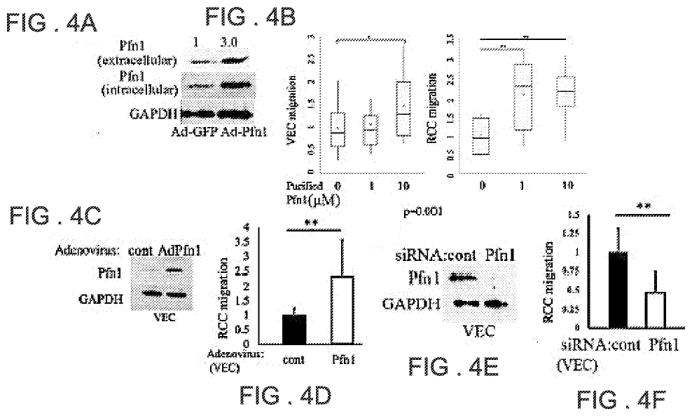

Given our observation of Pfn1 overexpression in TA-VEC, we next analyzed the effect of intracellular elevation of Pfn1 on its extracellular release based on immunoblot analyses of Pfn1 in conditioned media isolated from Pfn1-overexpressing vs. control VEC. We observed a dramatic increase in extracellular release of Pfn1 from VEC following transduction with an adenovirus-encoding Pfn1 (Ad-Pfn1; with Ad-GFP transduced cells serving as a control; FIG. 4A). The fold-change in the basal extracellular release of Pfn1 (˜3-fold) paralleled the level of gross protein overexpression (˜4.7-fold) based on cell lysate analyses. To determine whether soluble Pfn1 modulates cell migration, we next analyzed the effect of adding recombinant Pfn1 (rPfn1; 1 or 10 μM) to cultures of VEC or RCC cells. In both cases, rPfn1 stimulated cell migration, although RCC cells exhibited greater responsiveness vs. VEC, as 1 μM rPfn1 was sufficient to increase RVN motility by >2-fold, while VEC required 10 μM rPfn1 to increase motility by even ˜1.5 fold (FIG. 4B). Furthermore, in a transwell culture system, RVN cell motility was found to be >2.5-fold greater when allowed to migrate toward conditioned media harvested from the culture of Pfn1-overexpressing VEC vs the same from control VEC (FIG. 4C-D). Conversely, transwell migration of RVN cells toward VEC-conditioned media was reduced by ˜50% when Pfn1 expression was silenced in VEC (FIG. 4E). Collectively, these findings suggest that Pfn1 may serve as a paracrine VEC-secreted extracellular regulator of RCC cell migration.

### Pfn1-Actin Interaction Inhibitor Reduces RCC Cell Aggressiveness

We recently identified two structurally similar first generation small molecule antagonists of the Pfn1-actin interaction (C1: 8-(3-hydroxyphenyl)-10-(4-methylphenyl)-2,4,5,6,7,11,12-heptaazatricyclo[7.4.0.0³,⁷]trideca-1(13),3,5,9,11-pentaen-13-ol], and C2: 8-(3-hydroxyphenyl)-10-phenyl-2,4,5,6,7,11,12-heptaazatricyclo[7.4.0.0³,⁷]trideca-1(13),3,5,9,11-pentaen-13-ol]) (Structure-based virtual screening identifies a small-molecule inhibitor of the profilin 1-actin interaction, J. Biol. Chem. (2018) 293(7) 2606-2616).

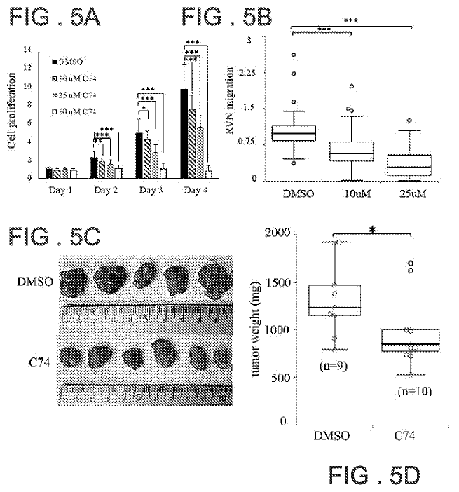

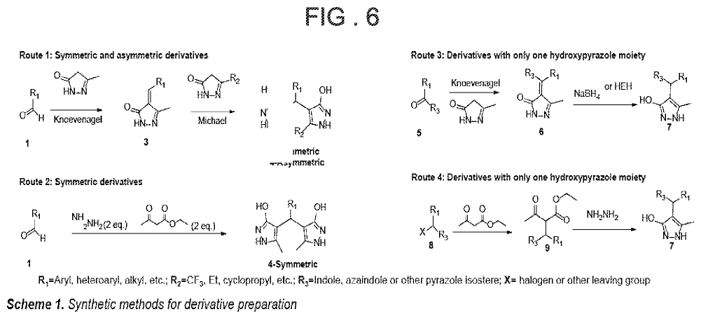

C74 (4-[(4-bromophenyl)(5-hydroxy-3-methyl-1Hpyrazol-4-yl)methyl]-3-methyl-1H-pyrazol-5-ol) is another compound that we recently discovered computationally that is able to reverse Pfn1's effect on actin polymerization in vitro but does not significantly alter actin polymerization on its own. Treatment with C74 reduced migration and proliferation of RVN cells in a dose-dependent manner; importantly, C74 was able to elicit these effects at a substantially lower range of concentrations (10-25 μM) than needed for C2 (FIGS. 5A-B).

To determine whether C74 has any therapeutic benefit in vivo, we performed an exploratory study in which we established subcutaneous tumors by transplantation of RENCA cells in syngeneic Balb/c mice and injected either C74 or DMSO (vehicle) directly at the tumor cell inoculation site daily over a course of 19 days starting from day 1. Based on the tumor weight measurements, the average end-point tumor burden of C74-treated animals (978.8±366.0 mg; n=10 animals) was found to be significantly lower than that of DMSO-treated animals (1327.5±397.5 mg; n=9 animals) (FIG. 5C-D). Collectively, these results demonstrate the ability of the C74 inhibitor of Pfn1-actin interaction to diminish RCC cell aggressiveness in vitro and in vivo.

### Pfn1 is a Clinically Relevant and Viable Target for Therapeutic Intervention Against Pathological Angio Genesis in Ocular Settings

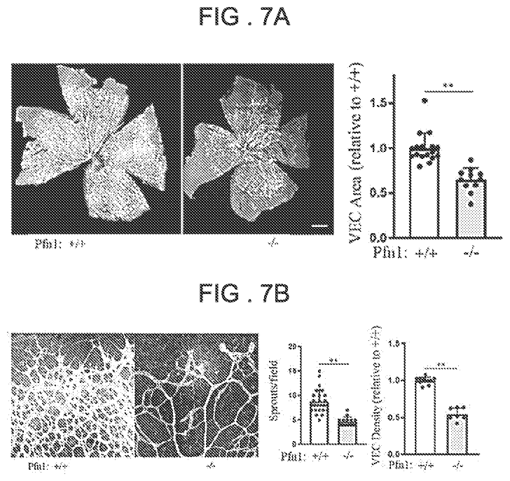

VEC migration is a fundamental aspect of angiogenesis. EC activation by pro-angiogenic cues allows a selective population of VEC (tip cells) to extend F-actin-rich filopodial protrusions (FLP), migrate toward guidance cues, and initiate vessel sprouting, while VEC trailing behind the tip cells (known as stalk cells) proliferate to elongate the sprouts. Dynamic remodeling of the actin cytoskeleton in response to angiogenic stimuli lies at the heart of tip cell migration. Pfn1, a G-actin binding protein, promotes actin polymerization in cells because of its ability to (a) promote nucleotide-exchange (ADP-to-ATP) on actin, and (b) interact with and stimulate actin polymerization by various polyproline (PLP)-domain bearing actin assembly factors that regulate actin filament dynamics at the zones of membrane protrusion (e.g. formins, Ena/VASP proteins). Pfn1-actin interaction in ECs is stimulated by VEGF through a site-specific (Y129) phosphorylation of Pfn1 that also allows Pfn1 to sequester ubiquitin ligase VHL causing HIF1 stabilization and angiocrine factor (VEGF, bFGF, Hb-EGF) production in a HIF1-dependent manner. Genetic disruption of Pfn1 phosphorylation reduces angiogenesis during tissue repair and tumor progression, suggesting Pfn1's regulation is important for angiogenesis in physiological and pathological settings. Consistent with the angiogenesis-promoting activity of Pfn1 and our findings that Pfn1 depletion leads to reduced angiogenic activity of VEC in vitro and ex vivo, we have demonstrated Pfn1 dependency for developmental retinal angiogenesis using a tamoxifen-inducible EC-specific conditional knockout (KO) mouse model (FIG. 7). Pfn1 is a clinically relevant anti-angiogenesis target because Pfn1 has an established link with vascular related pathology in diabetes. Specifically, Pfn1 expression is elevated in VEC in diabetes and in response to factors that promote diabetes-associated pathology (e.g. atherosclerosis, advanced glycation end products) in vitro and in vivo (including human patients). Pfn1 elevation exacerbates VEC dysfunction in diabetic milieu, and, conversely, Pfn1 depletion offers protection against oxidative damage, inflammation, and VEC dysfunction in diabetic animals in vivo. Furthermore, consistent with increased Pfn1 expression in EC during capillary morphogenesis in vitro, we have found that endothelial Pfn1 expression is elevated in experimentally induced abnormal angiogenesis in mouse retina and certain angiogenesis-associated human diseases including PDR and clear cell renal cell carcinoma.

With the exception of global absence of Pfn1 at the embryonic level, complete loss of Pfn1 is tolerable in many tissues in mammals. Second, we have also found that if Pfn1 disruption in EC is induced after birth, animals are viable and do not exhibit any discernible complications even up to adult life. Third, disruption of all key ligand interactions of Pfn1 also does not cause lethality in cultured cells including EC. Since these observations suggest differentiated cells tolerate loss of Pfn1 function reasonably well, Pfn1 inhibition will likely not result in systemic toxicities. Therefore, Pfn1 is a viable target for therapeutic intervention.

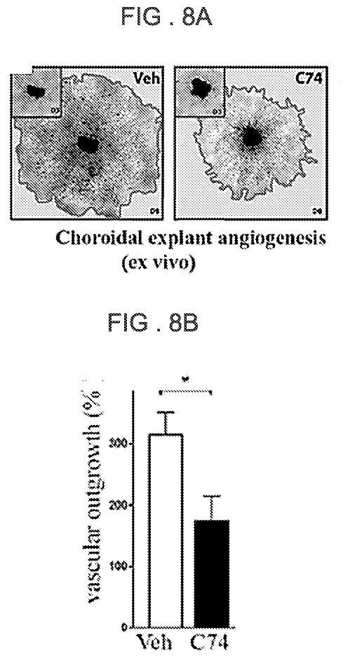

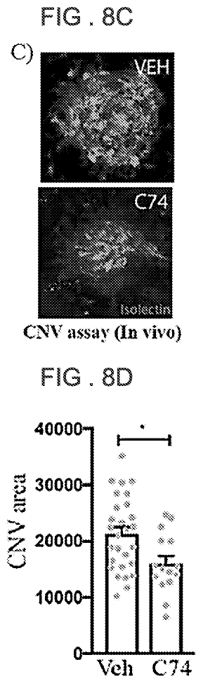

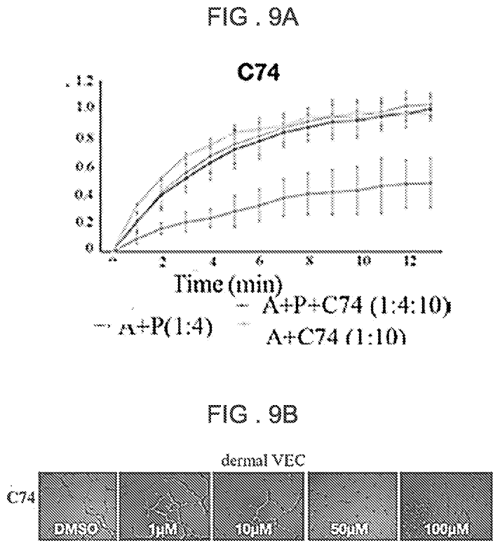

Consistent with our finding that actin:Pfn1 interaction is critical for angiogenic activity of VEC in vitro, we have demonstrated antiangiogenic effect of compound C74 targeting Pfn1-actin interaction in the settings of retinal and sub-retinal (choroidal) NV (FIG. 8). Collectively, these in vitro, ex vivo and in vivo findings supporting Pfn1-dependency for NV along with clinical correlates provide a strong scientific premise for the inhibition of the Pfn1-actin interaction to address diseases of aberrant angiogenesis in the eye.

### Anti-Angiogenic Effect of C74 in Tumor-Angiogenesis Setting

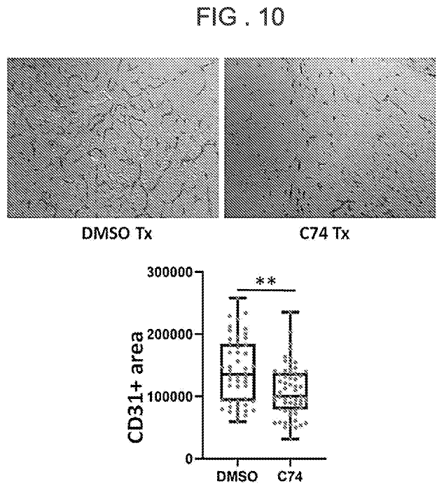

FIG. 10 shows that daily intratumoral administration of C74 reduces tumor angiogenesis (marked by lower CD31-positive immune-reactive region of tumor histosections) in subcutaneous tumors established by RENCA cells in Balb/c mice, relative to DMSO (vehicle) control.

### Materials and Methods

Cell culture, transfection and viral transduction: Murine VHL-deleted variant of RENCA (referred to as RVN cells) cells were cultured in DMEM with 10% (v/v) FBS and 1% (v/v) antibiotics. Human VHL-negative 786-0 cells (source: ATCC) were cultured in RPMI-1640 media supplemented with 10% (v/v) FBS and antibiotics. HmVEC-1, a widely used immortalized human dermal microvascular EC line (ATCC; CRL-3243—referred to as VEC), were cultured in MCDB-131 (Life Technologies; Carlsbad, CA) growth medium supplemented with 10% (v/v) FBS, 1% (v/v) antibiotics, 10 ng/mL EGF, 1 μg/mL Hydrocortisone and 10 mM L-Glutamine. For adenovirus infection, cells were plated, allowed to attach and then infected with adenovirus encoding either GFP (Ad-GFP) or Pfn1 (Ad-Pfn1) at a MOI of 500 for 24 h, before further incubation for an additional 48h in fresh culture media prior to use. For knockdown studies, cells were transfected with 50 nM of either smart-pool control or Pfn1 siRNA for 72 h.

Cell migration and proliferation assays: For single-cell migration experiments, RVN, VEC and 786-0 cells were plated in 24-well plates coated with type I collagen (Millipore) overnight. Where indicated, purified Pfn1 protein (Abcam, ab87760) was added to the culture media (at either 1 or 10 μM concentration) for several hours prior to time-lapse imaging. Time-lapse images of randomly migrating cells were collected using a 10× objective for 120 minutes at 1-minute time intervals using MetaMorph (Universal Imaging) software. The centroid of the cell nucleus was tracked using ImageJ and the average speed of migration was computed on a per-cell basis. For transwell migration experiments, 25,000 RVN cells were plated in triplicate in the upper chamber of 8.0 μM transwell plates in the serum-free media, and allowed to migrate either towards a 10% FBS gradient or VEC-conditioned media established in the lower chamber for 20 h (control wells contained serum-free media in the lower chamber as well). Non-migrating cells in the upper chamber were removed by swabbing; transmigrated cells were fixed in 3.7% formaldehyde and then stained with DAPI. Images were acquired at 10 random fields at 10× magnification for enumeration of nuclei using ImageJ. For cell proliferation assay, one thousand RCC cells were plated per well in 96-well plates using quadruplicate determinations, with proliferation assessed by time-course analyses of DAPI-stained nuclei based on images acquired over 3 to 5 random fields per well.

Conditioned media: Conditioned media were collected from the culture dishes following overnight incubation of cells in serum-free media. The collected media were filtered (0.45 μm size) and concentrated using a 10-kDa cutoff filter. The concentrate was reconstituted with 2× Laemmli sample buffer and boiled before being analyzed by gel electrophoresis.

Protein extraction and immunoblotting: Cell lysates were prepared by a modified RIPA buffer (25 mM Tris-HCL: pH 7.5, 150 mM NaCl, 1% (v/v) NP-40, 5% (v/v) glycerol), 1 mM EDTA, 50 mM NaF, 1 mM sodium pervanadate, and protease inhibitors supplemented with 6× sample buffer diluted to lx with the final SDS concentration in the lysis buffer was equivalent to 2%. Working dilutions for the various antibodies were: monoclonal Pfn1 (Abcam, ab124904; 1:3000), monoclonal Tubulin (Sigma, T9026, 1:3000) and monoclonal GAPDH (DSHB, DSHB-hGAPDH-2G7; 1:100).

Immunohistochemistry: For Pfn1 IHC of RCC TMA, tissue sections were deparaffinized and rehydrated tissue sections were incubated overnight with the primary anti-Pfn1 antibody (clone EPR6304, catalog #ab124904, Abcam, dilution 1:100). For immunodetection of the primary antibody, a secondary biotin-labeled anti-rabbit antibody (catalog #ab97049, Abcam) and streptavidinperoxidase conjugates (catalog #11089153001, Roche) were used. The staining was detected with the Pierce DAB Substrate Kit (catalog #34002, ThermoFisher). The slides were counterstained with hematoxylin before dehydration and mounting.

RCC TMA: The ccRCC TMA was obtained from the tissue bank of the National Center for Tumor Diseases, University of Heidelberg and used in accordance to the regulations of the tissue bank as well as under approval of the Ethics Committee of the University of Heidelberg School of Medicine. RCC specimens from 417 patients were included in the final analysis. The TMA specimen were collected between 1990 and 2005 with a median follow-up time of 112.7 months (range, 0.6-345.9 months). Clinicopathological features were: grade—G1/G2 (n=355, 85.1%), G3/4 (n=57, 13.7%), G unknown (n=5, 1.2%); stage—pT1/T2 (n=280, 67.2%), pT3/T4 (n=135, 32.4%), pTx (n=2, 0.5%); lymph node involvement—c/pN0 (n=393, 94.3%), N+(n=24, 5.8%); distant metastasis—cM0 (n=357, 85.6%), M+(n=60, 14.4%).

Transcriptome analysis of flow-sorted TA-VEC and NAT(normal adjacent tumor)-VEC. Surgically excised ccRCC tumor and tumor-adjacent normal kidney tissues were minced, digested with collagenase, passed through 70 mm mesh filter and ficoll density gradient before being stained with fluorescent-labeled antibodies against human CD34, CD45, and CD146 (all from BD Biosciences) and DAPI (Sigma-Aldrich). Stained cells were then sorted using a biocontained FACSAria cell sorters (in the Department of Immunology's Unified Flow Cytometry Facility, University of Pittsburgh) based on phenotype:

i.e. CD34+CD45negCD146+ VEC and CD34negCD45negCD146+ pericytes. RNA was isolated from the sorted cells and analyzed using Affymetrix U133 Plus 2.0 Array chips.

In vivo tumorigenesis: In 6 to 8 week old BALB/cJ mice, 1×106 RENCA cells were implanted subcutaneously in a 1:1 PBS:Matrigel (R&D Systems; Minneapolis, MN) mixture, supplemented with 81 μg C74 or DMSO control. Intratumoral injections were then given daily with either 320 μg C74 or control dissolved in saline, through day 19 post-implantation. On day 20, mice were sacrificed and harvested tumors were weighed. All animal experiments were performed according to the institutionally approved animal welfare guidelines.

Statistics: Statistical tests were performed with either one-way ANOVA followed by Tukey's post-hoc test or Mann-Whitney test when appropriate. Differences exhibiting p<0.05 were considered as statistically significant.

In view of the many possible embodiments to which the principles of the disclosed invention may be applied, it should be recognized that the illustrated embodiments are only preferred examples of the invention and should not be taken as limiting the scope of the invention.

1. A method for treating an angiogenesis-dependent disease or condition in a subject, comprising administering to the subject in need thereof a therapeutically effective amount of a compound of structure: 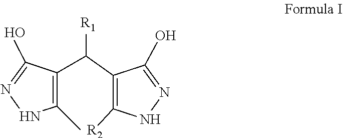
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₂ is hydrogen, alkyl, substituted alkyl, cycloalkyl, or substituted cycloalkyl; or 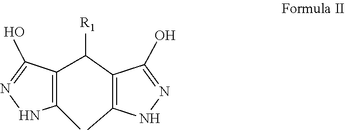
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; or 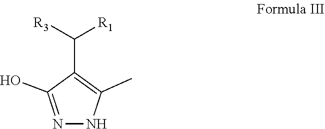
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₃ is N-heterocyclic group or substituted N-heterocyclic group,
thereby treating the angiogenesis-dependent disease or condition.

2. The method of claim 1, wherein the angiogenesis-dependent disease or condition is characterized by aberrant angiogenesis in the retina.

3. A method for treating an ocular disease or condition in a subject, comprising administering to the subject in need thereof a therapeutically effective amount of a compound of structure: 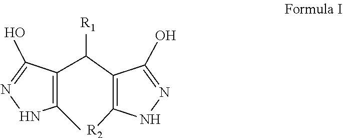
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₂ is hydrogen, alkyl, substituted alkyl, cycloalkyl, or substituted cycloalkyl; or 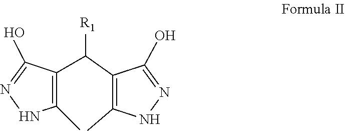
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; or 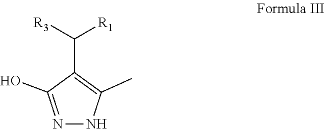
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₃ is N-heterocyclic group or substituted N-heterocyclic group,
thereby treating the ocular disease or condition.

4. The method of claim 3, wherein the ocular disease or condition is proliferative diabetic retinopathy, wet age-related macular degeneration, or retinopathy of prematurity.

5. A method for treating cancer in a subject, comprising administering to the subject in need thereof a therapeutically effective amount of a compound of structure: 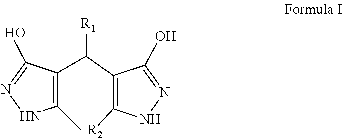
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₂ is hydrogen, alkyl, substituted alkyl, cycloalkyl, or substituted cycloalkyl; or 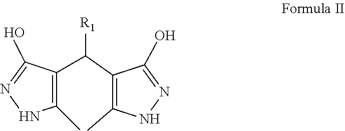
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; or 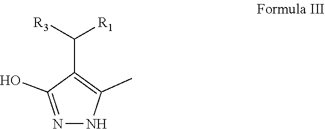
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₃ is N-heterocyclic group or substituted N-heterocyclic group,
thereby treating the cancer.

6. The method of claim 5, wherein the cancer is renal cell carcinoma.

7. The method of claim 5, wherein the cancer is clear cell renal cell carcinoma.

8. A method for inhibiting migration and/or proliferation of renal cell carcinoma cells, comprising contacting renal cell carcinoma cells with a compound of structure: 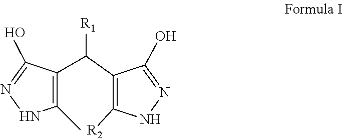
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₂ is hydrogen, alkyl, substituted alkyl, cycloalkyl, or substituted cycloalkyl; or 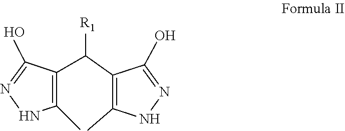
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; or 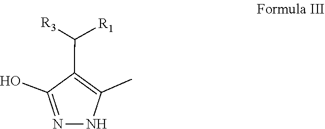
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₃ is N-heterocyclic group or substituted N-heterocyclic group.

9. The method of claim 4, wherein the renal cell carcinoma cells are RVN cells.

10. A method for inhibiting ocular neovascularization in a subject, comprising administering to the subject in need thereof a therapeutically effective amount of a compound of structure: 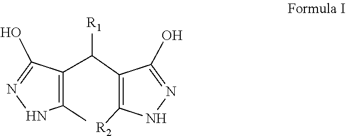
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₂ is hydrogen, alkyl, substituted alkyl, cycloalkyl, or substituted cycloalkyl; or 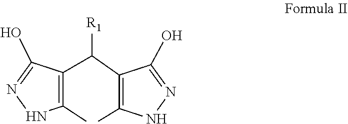
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; or 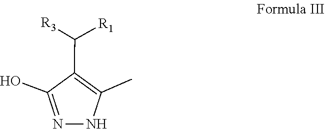
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₃ is N-heterocyclic group or substituted N-heterocyclic group.

11. A method for inhibiting tumor neovascularization in a subject, comprising administering to the subject in need thereof a therapeutically effective amount of a compound of structure: 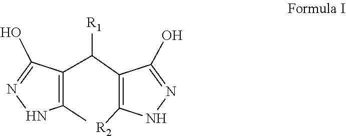
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₂ is hydrogen, alkyl, substituted alkyl, cycloalkyl, or substituted cycloalkyl; or 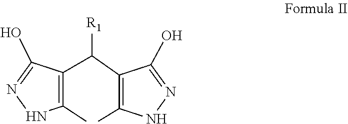
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; or 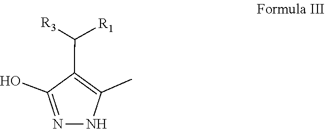
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₃ is N-heterocyclic group or substituted N-heterocyclic group.

12. A method for inhibiting tumor growth in a subject, comprising administering to the subject in need thereof a therapeutically effective amount of a compound of structure: 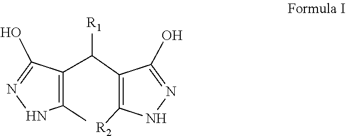
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₂ is hydrogen, alkyl, substituted alkyl, cycloalkyl, or substituted cycloalkyl; or 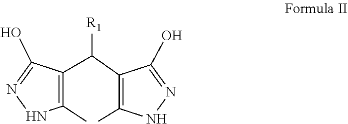
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; or 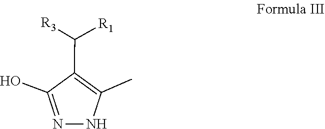
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₃ is N-heterocyclic group or substituted N-heterocyclic group,
thereby inhibiting tumor growth.

13. The method of claim 1, wherein the compound is:

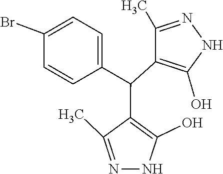 [SMILES: Cc1n[nH]c(O)c1C(c1ccc(Br)cc1)c1c(C)n[nH]c1O]

14. A compound, or a pharmaceutically acceptable salt thereof, of formula 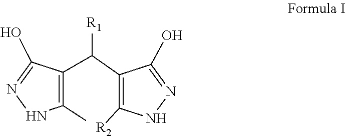
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₂ is hydrogen, alkyl, substituted alkyl, cycloalkyl, or substituted cycloalkyl; or 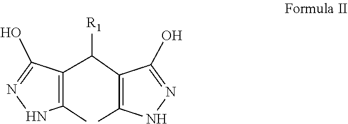
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; or 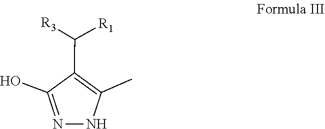
wherein R₁ is aryl, substituted aryl, heteroaryl, substituted heteroaryl, alkyl, substituted alkyl, heterocycloalkyl, substituted heterocycloalkyl, cycloalkyl, or substituted cycloalkyl; and
R₃ is N-heterocyclic group or substituted N-heterocyclic group,
provided that the compound is not 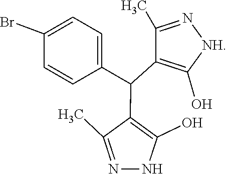 [SMILES: Cc1n[nH]c(O)c1C(c1ccc(Br)cc1)c1c(C)n[nH]c1O]
